(19) United States
(12) Patent Application Publication
(10) Pub. No.: US 2005/0279649 A1
(43) Pub. Date: Dec. 22, 2005

(54) Carrying case for dental appliance accessories

(76) Inventor: James N. Thacker, Houston, TX (US)

Correspondence Address: Hugh R. Kress;Suite 1800, 5718 Westheimer, Houston, TX, 77057, US

(21) Appl. No.: 10/870,363

(22) Filed: Jun. 17, 2004

(51) Int. Cl. A61B 19/02

(52) U.S. Cl. 206/63.5; 206/83

(57) ABSTRACT

A denture accessories carrying case having a form factor which belies its true function. In one embodiment, a carrying case comprises a container portion and a lid in a hinged, “clam-shell” relationship. The carrying case is configured to be of a size and shape that bears at least a passing resemblance to a common, everyday item not normally associated with dentures or denture accessories. In one embodiment, the carrying case is configured to resemble a typical cellular telephone, and is capable of containing a denture cleaning brush having a foreshortened handle alongside a small tube of denture adhesive.

### FIELD OF THE INVENTION

The present invention relates generally to the field of dental appliances, and more particularly relates to a carrying case for dental appliance accessories.

### BACKGROUND OF THE INVENTION

There are an estimated 32 million Americans who wear full or partial dentures or similar dental appliances. Dentures have been around for many years. The earliest form of denture was introduced more than 2,000 years ago. Today, dentures are of better quality and are more comfortable than ever before.

Replacing missing teeth has substantial benefits for one's health and appearance. A complete denture, also called a full denture, replaces all the natural teeth and provides support for cheeks and lips. Without this support, sagging facial muscles can make a person appear older. And by replacing missing teeth, dentures improve a person's ability to speak and to eat.

A denture is a removable replacement for missing teeth and adjacent tissues. It is most commonly made of acrylic resin (a plastic), sometimes in combination with various metals. Complete dentures replace all the teeth, while a partial denture fills in the spaces created by missing teeth and prevents other teeth from changing position. Complete dentures are either “conventional” or “immediate”. A conventional denture is placed in the mouth after all of the teeth are removed to allow for proper healing, whereas an immediate denture is placed as soon as the teeth are removed. The drawback behind an immediate denture is that it may require more adjustments after healing has taken place. An overdenture is a denture that is constructed with special attachments to attach it to some retained roots or dental implants so that it will stay firmly in place.

Like natural teeth, dentures must be brushed frequently to remove food deposits and plaque. Brushing helps prevent dentures from becoming permanently stained and helps a wearer's mouth stay healthy. It is generally regarded as best to use a brush specially designed for cleaning dentures. Denture cleaning brushes are commonly available in most drug stores and the like, and have a size and form factor substantially similar to a typical toothbrush, i.e., a handle eight to ten inches long with bristles disposed at one end of the handle.

Some denture wearers use hand soap or mild dishwashing liquid, which are both acceptable for cleaning dentures. Using other powdered household cleansers, which may be too abrasive, is to be avoided. The first step in cleaning dentures is to rinse away loose food particles thoroughly. The brush is then moistened and denture cleanser is applied.

Dentures may lose their shape if they are allowed to dry out. When they are not worn, dentures should be placed in a denture cleanser soaking solution or in water. Dentures should never be placed in hot water, which can cause them to warp. Ultrasonic cleaners may also be used to care for dentures. However, using an ultrasonic cleaner does not replace a thorough daily brushing.

Those of ordinary familiarity with dentures and similar dental prosthetics will understand that there are certain accessory items that are either convenient or essential to have available throughout the normal course of daily life. In order to be able to engage in routine daily activities, such as eating and drinking, it is often necessary for denture wearers to periodically maintain and/or adjust their dental appliances using various accessory items and applications, such as dental cleaning brushes, dental adhesive, and so on.

When at home or in other personalized environments, it may not be problematic for a denture wearer to ensure that he or she has ready access to the necessary accessories commonly required by denture wearers on a daily basis. However, when a denture wearer is faced with a situation in which they must venture away from personal spaces for extended periods of time (e.g., business travel, vacations, or the like), it is necessary to carry the requisite accessories along, whether in a briefcase, a travel bag, or on their person, such as in a pocket, handbag or the like. Without ready access to certain essential accessories at all times, a denture wearer's freedom to engage in daily activities can be significantly impaired and inconvenienced.

A typical denture cleansing brush has the general dimensions and form factor of an ordinary toothbrush, ordinarily being eight to ten inches long. Likewise, a typical tube of denture adhesive has a size and form factor comparable to a typical full-sized tube of toothpaste. These items are not conveniently stowed and carried in one's pockets. Moreover, after use, a denture cleansing brush will typically be wet, making it undesirable and possibly unhygienic to carry among other personal effects, be it in one's pocket, briefcase, handbag, or luggage.

Many studies have shown that the need for and wearing of dentures can have a seriously deleterious effect on one's self esteem. See D. M. Davis et al., “The Emotional Effects of Tooth Loss: A Preliminary Quantitative Study,” British Dental Journal, vol. 188, no. 9, May 13, 2000. It is well established that people with dentofacial abnormalities experience social consequences including greater degrees of social avoidance and being perceived as possessing negative personality characteristics. See, J. T. Newton et al., “A Preliminary Study of the Impact of Loss of Part of the Face and its Prosthetic Restoration,” Journal of Prosthetic Dentistry, 1999;82:585-590. Among the scholarship on the subject, the main themes identified in relation to the emotional impact of tooth loss include: loss of self confidence, concerns over appearance and self-image, tooth loss as a taboo subject which should not be discussed with other people, the desire to keep tooth loss a secret, and, notably, the need for prosthodontic privacy. See, e.g., J. Fiske et al., “The Emotional Effects of Tooth Loss,” The Proceedings of the European Prosthodontic Association 1997; 21:24; see also, A. Leao, “The Development of a Socio-Dental Measure of Dental Impacts on Daily Living,” Community Dental Health 1995; 13:22-26.

Given the widely-recognized social stigma associated with dental prosthetics, including dentures, it comes as no surprise that those requiring dental prosthetics such as dentures feel the need to conceal their edentulous condition. However, this can sometime be difficult to accomplish. A significant problem in this regard is that it is typically necessary for a wearer of dental prosthetics to have immediate access to certain accessory items at a moment's notice. The need to maintain and/or adjust one's dentures, for example, can and often does arise unexpectedly and without notice.

On the other hand, it is impractical or at least inconvenient to have dental maintenance accessories on hand at all times. At a minimum, a denture wearer should preferably have on hand a denture cleaning brush and a supply of denture adhesive, neither accessory being particularly convenient to carry on one's person on a routine basis. More importantly, the likelihood of others taking notice of such items being carried on one's person is highly undesirable, since the possession of such accessories as denture adhesive and denture cleaning brushes instantly reveals to others that one is a denture wearer.

### SUMMARY OF THE INVENTION

In view of the foregoing considerations, the present invention is directed to a carrying case for denture accessory items which provides not only addresses the practical considerations associated with having such items on hand at all times, but also addresses the aesthetic and social ramifications of doing so.

In accordance with one aspect of the invention, a carrying case for denture accessories is provided that is specifically configured and designed to disguise its true function. In one embodiment, a carrying case for denture accessories is designed to resemble, at least superficially, a typical cellular telephone. Thus, upon casual observation, the true function of the carrying case is not immediately apparent to onlookers. This enables denture wearers to keep the accessories in their possession without revealing the fact that they are, in fact, wearing dentures.

In accordance with another aspect of the invention, the denture accessories to be housed within the carrying case are modified by being reduced in size compared with conventional accessories. The diminished size of the accessories enables the carrying case to be of a size that would not be capable of containing conventional denture accessories, thereby furthering the element of disguise.

### BRIEF DESCRIPTION OF THE DRAWINGS

The foregoing and other features and advantages of the present invention will be best understood with reference to a detailed description of a preferred embodiment of the invention, which follows, when read in conjunction with the accompanying drawings, wherein:

FIG. 1 is a perspective view of a denture accessories carrying case in accordance with one embodiment of the invention, with its lid closed;

FIG. 2 is a perspective view of the carrying case of FIG. 1, with its lid open;

FIG. 3 is a perspective view of the carrying case of FIG. 1, with its lid partially open;

FIG. 4 is a top view of the lid from the carrying case of FIG. 1;

FIG. 5 is a side view of the lid from FIG. 4;

FIG. 6 is an end view of the lid from FIG. 4;

FIG. 7 is a top view of the base portion of the carrying case of FIG. 1;

FIG. 8 is a side view of the base portion from FIG. 7;

FIG. 9 is an end view of the base portion from FIG. 7;

FIG. 10 is a front view of a foreshortened denture cleaning brush in accordance with one embodiment of the invention;

FIG. 11 is a side view of the denture cleaning brush from FIG. 10;

FIG. 12 is a side view of a miniature tube of denture adhesive in accordance with one embodiment of the invention; and

FIG. 13 is a top view of the base portion of the carrying case from FIG. 1 showing the denture cleaning brush from FIG. 10 and the tube of denture adhesive from FIG. 12 disposed therein.

### DETAILED DESCRIPTION OF A SPECIFIC EMBODIMENT OF THE INVENTION

The disclosure that follows, in the interest of clarity, does not describe all features of actual implementations of the invention. It will be appreciated that in the development of any such actual implementations, as in any such project, numerous engineering decisions must be made to achieve the developers' specific goals and subgoals, which may vary from one implementation to another. Moreover, attention will necessarily be paid to proper engineering and practices for the environment in question. It will be appreciated that such an effort might be complex and time-consuming, but would nevertheless be a routine undertaking for those of ordinary skill in the relevant fields.

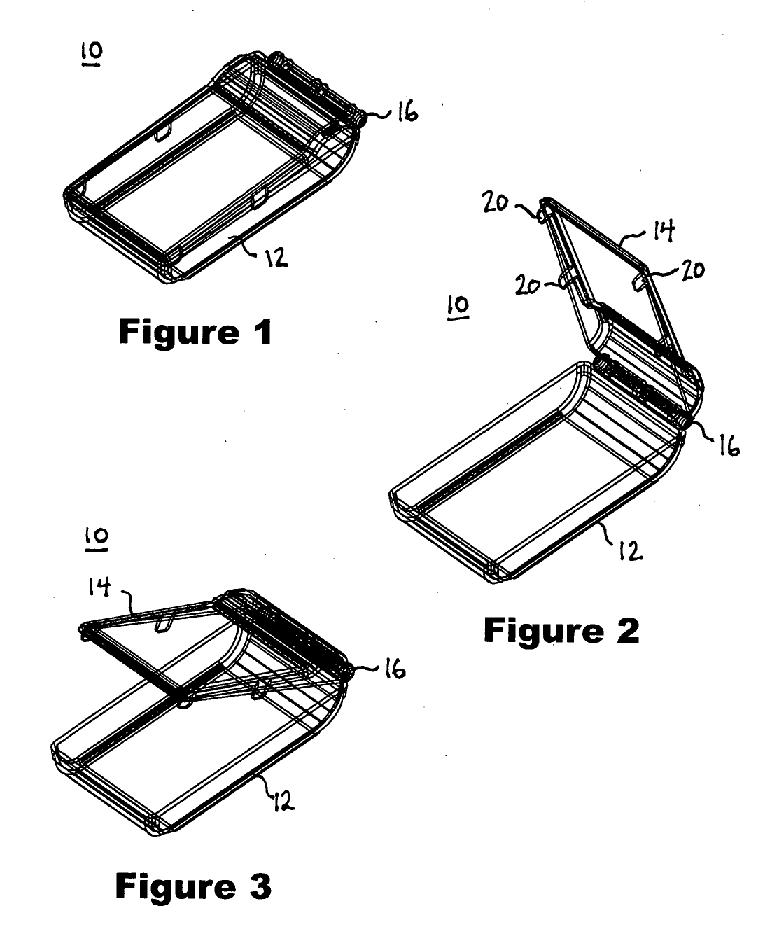

Referring to FIGS. 1, 2, and 3, there is shown a denture accessories carrying case 10 in accordance with one embodiment of the invention. As shown in these Figures, case 10 comprises a bottom containment portion 12 and a lid 14. A hinge 16 couples lid 14 to bottom portion 12 in a “clamshell” fashion. FIG. 1 shows case 10 in a closed state, FIG. 2 shows case 10 in an open state, and FIG. 3 shows case 10 in a partially-open state.

In one embodiment, case 10 has dimensions of approximately 3-½ inches (length) by 1⅞ inches (width) by 1 inch (height). This results in an inner volume of carrying case 10, with lid 14 closed, of approximately 1¾ cubic inches. The size of carrying case 10 is such that it can be conveniently carried on one's person, for example, in one's pocket or handbag or the like. Preferably, case 10 is made of a rigid plastic material, such as molded polypropylene or the like, as would be apparent to those of ordinary skill in the art.

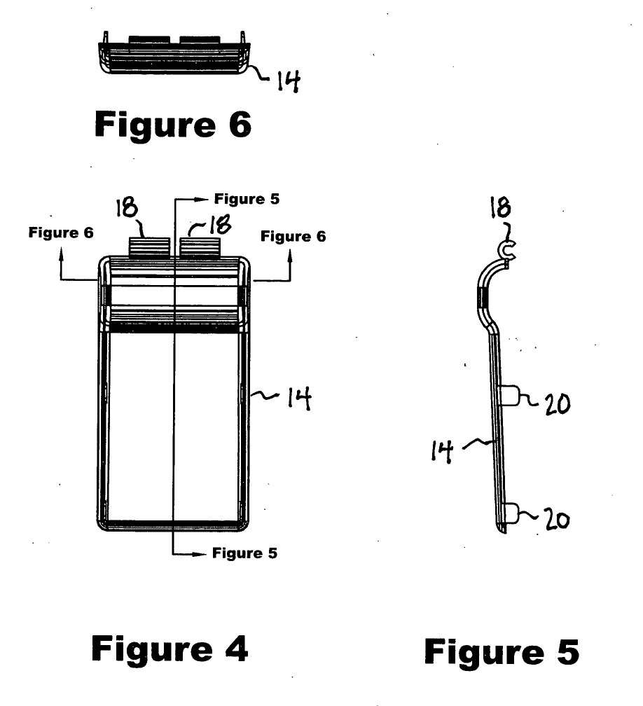

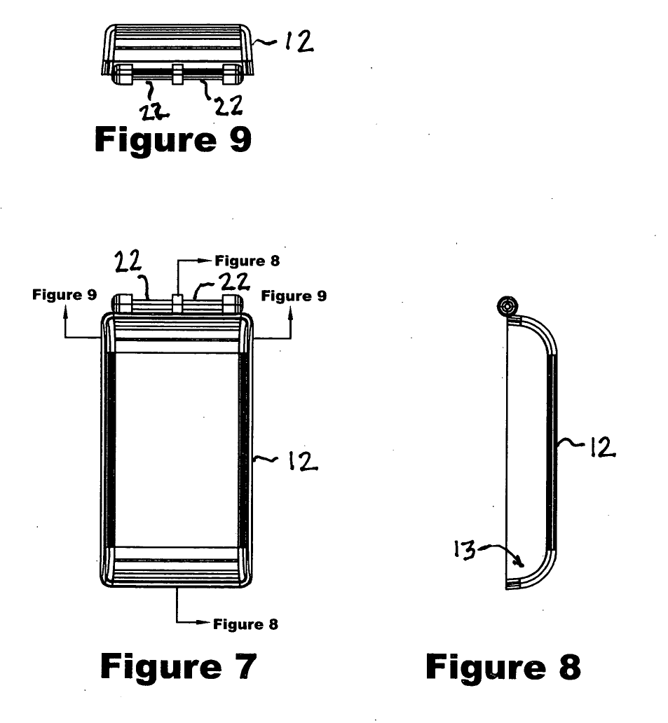

FIGS. 4, 5 and 6 show top, side, and end views, respectively, of lid 14. As can be seen in FIGS. 4 and 5, at one end of lid 14, a pair of hinge tubes 18 are formed. As will be apparent with reference to FIGS. 7, 8, and 9, hinge tubes 18 cooperate with hinge pins formed at one end of containment portion 12 to form hinge 16 which mates cover 14 and base 12.

Also formed around the perimeter of cover 14 are a plurality of blades 20 which serve to align cover 14 and base 12 when carrying case 10 is closed.

FIGS. 7, 8 and 9 are top, side, and end views, respectively, of base portion 12. As mentioned above, and as is especially apparent from FIG. 7, a pair of hinge pins 22 are formed at one end of base portion 12. Hinge pins 22 are adapted to be received in hinge tubes 18 to form hinge 16. In accordance with one aspect of the invention, hinge pins 22 are substantially cylindrical, except that there is formed on each hinge pin 22 a flat portion. Likewise, a corresponding flat portion on the inner surface of hinge tubes 18 is formed. As would be appreciated by those of ordinary skill in the art, the respective flats formed on hinge pins 22 and hinge tubes 18 are located such that they cooperate by coming into alignment with one another when cover 14 is folded down over top of containment portion 12. This arrangement results in a slight resistance to the opening of lid 14, thereby avoiding accidental opening of carrying case 10 in use.

From FIGS. 1 through 9, it is apparent to those of ordinary skill that carrying case 10 bears a passing resemblance to a conventional “flip-type” cellular telephone. At the very least, and in accordance with an important aspect of the invention, the outward appearance of carrying case 10 does not give a casual observer any cue as to its true function. Indeed, the size and shape of carrying case 10 actually subtly suggests that it is incapable of carrying a denture cleaning brush and a conventionally-sized tube of denture adhesive. This element of disguise is believed to be significant, inasmuch as it enables denture wearers to carry denture accessories, as will be described below in further detail, without making it immediately apparent to those around them that they are in possession of such items.

As used herein, the term “disguise” as applied to the outward appearance of carrying case 10, is intended to suggest, as just described, that such outward. appearance (size and shape) does not give casual observers any immediate cues that carrying case 10 is functioning to contain denture accessories, or, in fact, that carrying case 10 actually is a carrying case.

It is to be noted that the element of disguise can be, but need not be, carried to extremes. For example, in the disclosed exemplary embodiment, carrying case 10 is designed to bear a passing resemblance to a cellular telephone. However, this resemblance to a cellular phone arises primarily out of the form factor of carrying case 10, i.e., its size and general configuration. Additional misleading details, such as false phone keys or a false cellular phone display, need not, and in the disclosed embodiment, are not provided to achieve a sufficient element of disguise.

Furthermore, although the disclosed embodiment is configured to resemble a cellular phone, those of ordinary skill in the art having the benefit of the present disclosure will appreciate that carrying cases could be configured to resemble any one of a number of common, everyday items that casual observers would (1) not associate with the wearing of dentures or carrying of denture accessories; and (2) not deem unusual to see in a person's possession in the course of daily life. As long as these two features are present, it is believed that the objectives of the present invention can be successfully achieved.

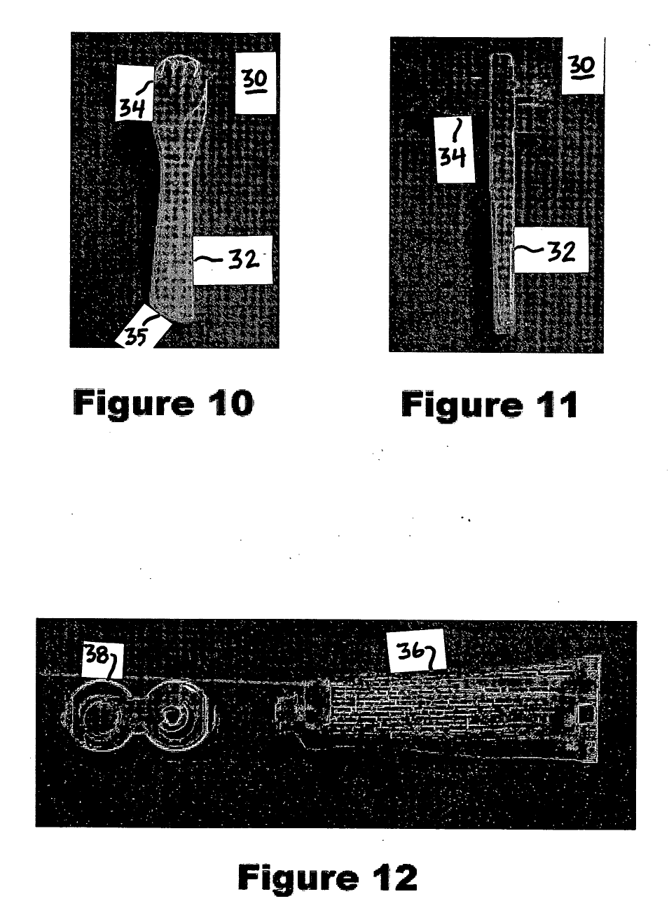

Turning to FIGS. 10 and 11, there are shown front and side views, respectively, of a denture cleaning brush 30 in accordance with one embodiment of the invention. As shown in FIGS. 10 and 11, brush 30 comprises a handle 32 and a two-sided brush head 34. Brush head 34 is sized and configured in a more or less conventional fashion, as would be familiar to most denture wearers. On the other hand, handle 32 is configured differently from conventional denture cleaning brushes, inasmuch as it is substantially foreshortened compared to conventional denture cleaning brushes. In the disclosed embodiment, handle 32 is approximately 3 5/16 inches in length, whereas conventional denture cleaning brushes have handles substantially longer, similar to everyday toothbrushes (i.e., on the order of eight to ten inches in length, for example).

The foreshortening of handle 32 is a significant feature, since it allows brush 30 to fit inside carrying case 10. This enhances the element of disguise of the present invention, since the size and form factor of carrying case 10 can be such that it would not be capable of housing a conventional denture cleaning brush. In the disclosed embodiment, and as shown in FIG. 10, the end 35 of handle 32 is contoured to conform generally with the internal curvature of containment portion 12 of carrying case 12, as designated generally with reference numeral 13 in FIG. 8.

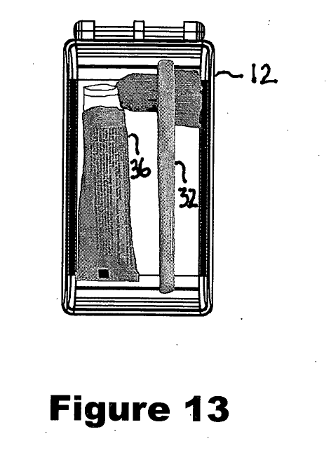

Turning to FIG. 12, there is shown a tube 36 for holding a supply of denture adhesive. In the disclosed embodiment, tube 36 is approximately three inches in length. It is contemplated that tube may be refillable from a larger, conventionally-sized tube of denture adhesive, and to this end, a screw-on nozzle (not shown) may be provided to facilitate the squeezing of adhesive from a larger tube to smaller tube 36 when refilling is required. A flip-top cap 38 for tube 36 is also shown in FIG. 12. In accordance with one aspect of the invention, tube 36 is sized, like handle 32 of brush 30, to be capable of fitting within carrying case 10 alongside brush 32, as shown in FIG. 13. It is to be noted for the sake of clarity that lid 14 is removed from containment portion 12 of case 10 in FIG. 13.

From the foregoing, it will be apparent to those of ordinary skill in the art that a carrying case for dental appliance accessories has been disclosed which is configured to disguise its true function, thereby enabling denture wearers to discreetly keep necessary accessories in their possession without revealing their status as denture wearers. Although specific embodiments of the invention have been discussed herein, it is to be understood that this has been done solely for the purposes of describing various aspects of the invention, and is not intended to be limiting with respect to the scope of the invention as defined by the claims that follow.

It is contemplated that various substitutions, alterations, and/or modifications, including but not limited to those design alternatives specifically mentioned herein, may be made to the disclosed embodiments without departing from the spirit and scope of the invention as defined in the claims. For example, and as noted above, although an embodiment of the invention in which the carrying case is designed to resemble a cellular telephone, those of ordinary skill in the art having the benefit of this disclosure would appreciate that a carrying case may be adapted to resemble, to varying degrees, a wide variety of other common objects not associated with dentures and denture accessories, and having form factors which would not suggest that they were capable of containing a complement of full-sized denture accessories.

1. A denture accessories carrying case, comprising:
a rigid housing comprising a containment portion and a lid, said containment portion and said lid being coupled together by a hinge;
wherein said rigid housing has a size and shape which disguises its function as a denture accessories carrying case.

2. A denture accessories carrying case in accordance with claim 1, wherein said rigid housing is configured to resemble an object not associated with dentures and denture accessories.

3. A denture accessories carrying case in accordance with claim 1, wherein said rigid housing is adapted to contain a foreshortened denture cleaning brush and a small tube of denture adhesive.

4. A denture accessories carrying case in accordance with claim 2, wherein said object is a cellular telephone.

5. A denture accessories carrying case in accordance with claim 1, wherein said case has a volume of less than approximately 2 cubic inches.

6. A denture accessories carrying case in accordance with claim 1, wherein said denture accessories comprise a denture cleaning brush and a tube of denture adhesive.

7. A denture accessories carrying case in accordance with claim 6, wherein said denture cleaning brush has a foreshortened handle by comparison to conventional denture cleaning brushes.

8. A denture accessories carrying case in accordance with claim 7, wherein said foreshortened handles is approximately 3½ inches in length.

9. A denture accessories carrying case in accordance with claim 8, wherein said tube of denture adhesive is approximately three inches in length.
